(19) United States
(12) Patent Application Publication
(10) Pub. No.: US 2012/0128670 A1
(43) Pub. Date: May 24, 2012

(54) mTOR INHIBITOR AND ANGIOGENESIS INHIBITOR COMBINATION THERAPY

(75) Inventors: Sharon M. Barr, Huntington, NY (US); Prafulla C. Gokhale, Superior, CO (US); Robert C. Wild, Carmal, IN (US)

(73) Assignee: OSI PHARMACEUTICALS, LLC, Farmingdale, NY (US)

(21) Appl. No.: 13/387,754

(22) PCT Filed: Jul. 30, 2010
(86) PCT No.: PCT/US10/43824

§ 371 Date: Jan. 30, 2012

Related U.S. Application Data
(60) Provisional application No. 61/230,579, filed on Jul. 31, 2009.

(51) Int. Cl. A61K 31/53; A61P 35/04; A61P 35/00; A61K 39/395; A61K 38/16

(52) U.S. Cl. 424/133.1; 514/243; 514/19.3; 514/19.8; 424/158.1

(57) ABSTRACT

Cancer therapy comprising treatment with an mTOR inhibitor, such as a dual mTORC1/mTORC2 inhibitor, such as OSI-027, in combination with an angiogenesis inhibitor.

This application claims priority of U.S. Appl. No. 61/230,579 (Jul. 31, 2009), the content of which is incorporated herein in its entirety.

### FIELD OF THE INVENTION

In some aspects, the present invention pertains to combination cancer therapies, including treating tumors or tumor metastases in a patient by administering simultaneously or sequentially a therapeutically effective regimen comprising an mTOR inhibitor and an angiogenesis inhibitor, e.g., to produce synergistic anti-tumor effects, with or without additional agents or treatments. In some aspects, invention is directed to a pharmaceutical composition comprising an mTOR inhibitor and an angiogenesis inhibitor, and optionally one or more other anti-cancer agents.

### BACKGROUND OF THE INVENTION

An angiogenesis inhibitor is a substance that inhibits angiogenesis (the growth of new blood vessels). It can be endogenous or can come from outside as a drug or a dietary component. Every solid tumor (in contrast to liquid tumors like leukemia) needs to generate blood vessels to keep it alive once it reaches a certain size. Usually, blood vessels are not built elsewhere in an adult body unless tissue repair is actively in process. The angiostatic agent endostatin and related chemicals can suppress the building of blood vessels, preventing the cancer from growing indefinitely. In tests with patients, tumors became inactive and stayed that way even after the endostatin treatment was finished. The treatment has very few side effects but appears to have very limited selectivity.

The Tor genes were originally identified in yeast as the targets of the drug rapamycin. The structurally and functionally conserved mammalian counterpart of yeast TOR, mTOR was later discovered. mTOR is a member of the phosphoinositide kinase-related kinase (PIKK) family, but rather than phosphorylating phosphoinositides, phosphorylates proteins on serine or threonine residues. Genetic studies have shown that mTOR is essential for cell growth and development in fruit flies, nematodes and mammals, and the disruption of the genes encoding mTOR results in lethality in all species. Several studies have demonstrated that mTOR has a central role in controlling cell growth, proliferation and metabolism. mTOR regulates a wide range of cellular functions, including translation, transcription, mRNA turnover, protein stability, actin cytoskeletal organization and autophagy. There are two mTOR complexes in mammalian cells. mTOR complex I (mTORC1) is a raptor-mTOR complex, which mainly regulates cell growth in a rapamycin-sensitive manner whereas mTOR complex II (mTORC2) is a rictor-mTOR complex, which regulates cytoskeletal organization in a rapamycin-insensitive manner.

The best-characterized function of mTOR in mammalian cells is regulation of translation. Ribosomal S6 kinase (S6K) and eukaryotic initiation factor 4E binding protein 1 (4E-BP1), the most extensively studied substrates of mTOR, are key regulators of protein translation. S6K is the major ribosomal protein kinase in mammalian cells. Phosphorylation of S6 protein by S6K selectively increases the translation of mRNAs containing a tract of pyrimidines motif; these mRNAs often encode ribosomal proteins and other translational regulators. Thus, S6K enhances overall translation capacity of cells. 4E-BP1, another well-characterized mTOR target, acts as a translational repressor by binding and inhibiting the eukaryotic translation initiation factor 4E (eIF4E), which recognizes the 5′ end cap of eukaryotic mRNAs. Phosphorylation of 4E-BP1 by mTOR results in a dissociation of 4E-BP1 from eIF4E, thereby relieving the inhibition of 4E-BP1 on eIF4E-dependent translation initiation. eIF4E overexpression enhances cell growth and transforms cells by increasing the translation of a subset of key growth-promoting proteins, including cyclin D1, c-Myc and VEGF. Therefore, mTOR-dependent regulation of both 4E-BP1 and S6K might be one mechanism by which mTOR positively regulates cell growth. mTOR integrates two of the most important extracellular and intracellular signals involved in the regulation of cell growth: growth factors and nutrients. Growth factor, such as insulin or IGF1 and nutrients, such as amino acids or glucose, enhance mTOR function, as evidenced by an increased phosphorylation of S6K and 4E-BP1. Rapamycin or dominant negative mTOR inhibits these effects, indicating that mTOR integrates the regulation of signals from growth factors and nutrients.

Rapamycin, a macrolide antibiotic has been shown to specifically inhibit mTOR kinase activity in vitro and in vivo in several studies. Although precise mechanism by which rapamycin inhibits mTOR function is not well understood, it is known that rapamycin first binds to FKBP12 (FK506 binding protein) and then binds to FRB domain of mTOR and thus inhibit mTOR activity by inducing conformational changes, which inhibits substrate binding. Rapamycin has been widely used as a specific mTOR inhibitor in preclinical studies to demonstrate role of mTOR in signal transduction and cancer. But rapamycin was not developed as a cancer therapy because of stability and solubility problems even though significant antitumor activity was observed in the NCI screening program. However, synthesis of rapamycin analogues with superior solubility and stability properties has led to run the clinical trails with CCI-779, RAD001 and AP23573. The most advanced rapamycin analogue, CCI-779 has shown modest anti-tumor activity in Phase II breast, renal carcinoma and mantle cell lymphoma clinical trials. CCI-779 (temsirolimus) has attained marketing approval in renal cell carcinoma.

Although rapamycin analogues are in clinical development for cancer as mTOR kinase inhibitors, the clinical outcome with CCI-779 is just modest in breast and renal cancer patients. This is probably because rapamycin partially inhibits mTOR function through raptor-mTOR complex (mTORC1). It has been also found that ⅔ of the breast cancer and V2 of renal cancer patients are resistant to rapamycin therapy. With a recent discovery of rictor-mTOR complex (mTORC2) which is involved in phosphorylation of AKT (S473) that is important in regulation of cell survival and modulation of PKCa that plays a major role in regulation of actin cytoskeletal organization in a rapamycin-independent manner, and inhibition of these activities of mTOR is probably important for broader antitumor activity and better efficacy. Therefore, it is desirable to use an mTOR inhibitor which would inhibit mTORC1 and mTORC2.

Signaling pathways that are upstream and downstream of mTOR are often deregulated in variety of cancers, including breast, lung, kidney, prostate, blood, liver, ovarian, thyroid, GI tract and lymphoma. High levels of dysregulated mTOR (mammalian target of rapamycin) activity are associated with variety of human cancers and several hamartoma syndromes, including tuberous sclerosis complex, the PTEN-related hamartoma syndromes and Peutz-Jeghers syndrome.

The activated mTOR pathway signaling regulates protein translation and VEGF secretion which promotes tumor growth, therefore, Inhibition of this pathway using an mTOR inhibitor in combination with an antiangiogenic inhibitor such as VEGF neutralizing antibody, receptor antagonists or VEGF receptor tyrosine kinase inhibitors may benefit the cancer treatment of patients. Furthermore, mTORC1 specific inhibitors such as rapamycin inhibit translation and activity of HIF1α thereby reducing VEGF expression. However, it also stimulates mTORC2-AKT signaling and AKT is important for VEGF-mediated angiogenesis. Therefore, a dual mTORC1/mTORC2 inhibitor which abolishes both HIF1α and AKT activity would inhibit angiogenesis. Thus a combination of mTORC1/mTORC2 inhibitor with VEGF inhibitors would result in better therapeutic benefit for the patients in cancer treatment.

Oncogenes including overexpressed receptor tyrosine kinases and constitutively activated mutant receptors activate PI3K-mediated signaling pathways. Additional alterations of the PI3K-mTOR pathway in human cancers include amplification of the p110 catalytic subunit of PI3K, loss of PTEN phosphatase function, amplification of AKT2, mutations in TSC1 or TSC2, and overexpression or amplification of eIF4E or S6K1. Mutation or loss of heterozygosity in TSC1 and TSC2 most often give rise to Tuberous Sclerosis (TSC) syndrome. TSC is rarely associated with malignant tumors, although patients with TSC are at risk for malignant renal cancer of clear-cell histology. Although inactivation of TSC might not lead to malignancy per se, deregulation of this pathway seems crucial for angiogenesis in developing malignancies. TSC2 regulates VEGF production through mTOR-dependent and -independent manner.

With the recent discovery of a rapamycin-independent function of mTOR (by mTOR2) in phosphorylation AKT (at S473), it is believed that inhibition of mTOR function by rapamycin is partial. Therefore, the use of a direct mTOR kinase inhibitor, which would completely inhibit the function of both mTORC1 and mTORC2, is required for broader anti-tumor activity and better efficacy.

Known angiogenesis inhibitors include the drug bevacizumab (Avastin) and sunitinib (Sutent). However, under some circumstances, anti-angiogenic therapy has been unsuccessful when angiogenesis inhibitors such as, for example, bevacizumab and sunitinib, are used as a monotherapy. The lack of success is believed to be caused by resistance to the monotherapy. In this regard, resistance refers to a neoplasm having cells that express a resistant mutant form of VEGF receptor or cells that overexpress a VEGF receptor.

Therefore, there is a need for new therapeutic treatments to overcome the resistance to anti-angiogenic monotherapy. The inventors of the present invention surprisingly discovered the use of combination of an mTOR inhibitor with an anti-angiogenic inhibitor provides a synergistic effect in treating tumors or tumor metastases in a patient.

### SUMMARY OF THE INVENTION

The present invention provides a method for treating tumors or tumor metastases in a patient, comprising administering to the patient simultaneously or sequentially a therapeutically effective amount of a combination of an mTOR inhibitor and an angiogenesis inhibitor that produces a synergistic anti-tumor effect, with or without additional agents or treatments, such as other anti-cancer drugs or radiation therapy. The present invention also provides a pharmaceutical composition comprising a pharmaceutically acceptable carrier or excipient and, as active ingredient, an mTOR inhibitor and an angiogenesis inhibitor, or a pharmaceutically acceptable salt thereof, and optionally one or more other anti-cancer agents.

In some aspects, the mTOR inhibitor can be the compound (1R,4R)-4-(4-amino-5-(7-methoxy-1H-indol-2-yl)imidazo[1,5-f][1,2,4]triazin-7-yl)cyclohexanecarboxylic acid (also named OSI-027). In some aspects, the OSI-027 is a tromethamine salt thereof.

In some aspects, the angiogenesis inhibitor can be bevacizumab (Avastin®) or sunitinib (Sutent®).

### BRIEF DESCRIPTION OF THE DRAWINGS

FIG. 1: In vivo efficacy study of OSI-027 or bevacizumab (Avastin®) alone and in combination of OSI-027 with bevacizumab (Avastin®) in DU145 prostate carcinoma xenografts.

FIG. 2: In vivo efficacy study of OSI-027 or bevacizumab (Avastin®) alone and in combination of OSI-027 with bevacizumab (Avastin®) in DU145 prostate carcinoma xenografts with extended dosing for 28 days.

FIG. 3: In vivo efficacy study of OSI-027 or sunitinib (Sutent®) alone and the combination of OSI-027 with sunitinib (Sutent®) in H292 lung carcinoma xenografts.

FIG. 4: In vivo efficacy study of OSI-027 or sunitinib (Sutent®) alone and the combination of OSI-027 with sunitinib (Sutent®) in the OVCAR-5 ovarian carcinoma xenografts.

FIG. 5: In vivo efficacy study of OSI-027 or bevacizumab (Avastin®) alone and in combination of OSI-027 with bevacizumab (Avastin®) in SKOV3 ovarian carcinoma xenografts.

### DETAILED DESCRIPTION OF THE INVENTION

In some aspects, the invention provides, a method of treating a tumor or tumor metastasis comprising treating a patient in need thereof with a regimen comprising administering an mTOR inhibitor and an angiogenesis inhibitor, wherein the regimen provides a therapeutically effective synergistic or additive effect; and wherein the mTOR inhibitor is a an agent as described below.

In some aspects, the mTOR inhibitor is a dual mTORC1 and mTORC2 inhibitor.

In some aspects, the mTOR inhibitor comprises OSI-027, which has the formula:

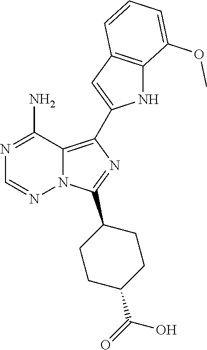

or a pharmaceutically acceptable salt thereof. In some aspects, the mTOR inhibitor comprises a tromethamine salt of OSI-027.

In some aspects, the mTOR inhibitor and the angiogenesis inhibitor behave synergistically.

In some aspects, the angiogenesis inhibitor comprises an inhibitor of VEGF activity. In some aspects, the angiogenesis inhibitor comprises bevacizumab, aflibercept, ABT-869, Axitinib, ranibizumab, or BIBF1120. In some aspects, the angiogenesis inhibitor comprises an anti-VEGF antibody. In some aspects, the angiogenesis inhibitor comprises bevacizumab or ranizumab. In some aspects, the angiogenesis inhibitor comprises a VEGFR inhibitor. In some aspects, the angiogenesis inhibitor comprises sunitinib, sorafenib, vatalanib, AMG-706, CP-547632, pazopanib, ABT-869, IMC-1C11, cediranib, KM-2550, 2C3, IMC-18F1, or OSI-930.

In some aspects, the mTOR inhibitor comprises OSI-027 and the angiogenesis inhibitor comprises bevacizumab or sunitinib.

In some aspects, the mTOR pathway is activated in the tumor. In some aspects, the tumor comprises ovarian, prostate, or non small cell lung cancer. In some aspects, the tumor comprises renal cell carcinoma, endometrial carcinoma, or glioblastoma. In some aspects, the tumor comprises lymphoma or pancreatic cancer. In some aspects, the tumor or tumor metastasis is insensitive or refractory to treatment with the angiogenesis inhibitor as a single agent.

In some aspects, the method results in improved survival of the patient as compared to treatment with either the mTOR or the angiogenesis inhibitor alone.

In some aspects, OSI-027 is administered in an amount of about 0.5 to about 5 mg/kg body weight on days of administration. In some aspects, OSI-027 and bevacizumab are administered in a mass ratio of about 6 to about 15. In some aspects, OSI-027 and sunitinib are administered in a mass ratio of about 0.2 to about 2.

In some aspects, one or more other anti-cancer agents is administered, such as cyclophosphamide, chlorambucil, cisplatin, busulfan, melphalan, carmustine, streptozotocin, triethylenemelamine, mitomycin C, methotrexate, etoposide, 6-mercaptopurine, 6-thiocguanine, cytarabine, 5-fluorouracil, capecitabine, dacarbazine, actinomycin D, doxorubicin, daunorubicin, bleomycin, mithramycin, alkaloids, paclitaxel, paclitaxel derivatives, cytostatic agents, glucocorticoids, corticosteroids, nucleoside enzyme inhibitors, or amino acid depleting enzymes.

In some aspects, there is provided a pharmaceutical composition comprising an mTOR inhibitor and an angiogenesis inhibitor according to claim 1. In some aspects, the composition comprises OSI-027. In some aspects, the composition comprises bevacizumab or sunitinib.

mTOR Inhibitor

In some aspects, the mTOR inhibitor is a compound of Formula (I)

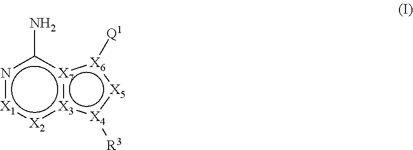

or a pharmaceutically acceptable salt thereof, wherein:

X₁, and X₂ are each independently N or C-(E¹)ₐₐ;

X₅ is N, C-(E¹)ₐₐ, or N-(E¹)ₐₐ;

X₃, X₄, X₆, and X₇ are each independently N or C;

wherein at least one of X₃, X₄, X₅, X₆, and X₇ is independently N or N-(E¹)ₐₐ;

R³ is C₀₋₁₀alkyl, cycloC₃₋₁₀alkyl, aminomethylcycloC₃₋₁₀alkyl, bicycloC₅₋₁₀alkyl, aryl, heteroaryl, aralkyl, heteroaralkyl, heterocyclyl or heterobicycloC₅₋₁₀alkyl any of which is optionally substituted by one or more independent G¹¹ substituents;

Q¹ is -A(R¹)ₘB(W)ₙ or —B(G¹¹)ₙA(Y)ₘ;

A and B are respectively, 5 and 6 membered aromatic or heteroaromatic rings, fused together to form a 9-membered heteroaromatic system excluding 5-benzo[b]furyl and 3-indolyl; and excluding 2-indolyl, 2-benzoxazole, 2-benzothiazole, 2-benzimidazolyl, 4-aminopyrrolopyrimidin-5-yl, 4-aminopyrrolopyrimidin-6-yl, and 7-deaza-7-adenosinyl derivatives when X₁ and X₅ are CH, X₃, X₆ and X₇ are C, and X₂ and X₄ are N;

or Q¹ is -A(R¹)ₘA(Y)ₘ, wherein each A is the same or different 5-membered aromatic or heteroaromatic ring, and the two are fused together to form an 8-membered heteroaromatic system;

R¹ is independently, hydrogen, —N(C₀₋₈alkyl)(C₀₋₈alkyl), hydroxyl, halogen, oxo, aryl(optionally substituted with 1 or more R³¹ groups), hetaryl(optionally substituted with 1 or more R³¹ groups), C₁₋₆alkyl, —C₀₋₈alkylC₃₋₈cycloalkyl, —C₀₋₈alkyl-NR³¹¹S(O)₀₋₂R³²¹, —C₀₋₈alkyl-NR³¹¹S(O)₀₋₂NR³²¹R³³¹, —C₀₋₈alkyl-S(O)₀₋₂NR³¹¹R³²¹, —C₀₋₈alkyl-NR³¹¹COR³²¹, —C₀₋₈alkyl-NR³¹¹CO₂R³²¹, —C₀₋₈alkyl-NR³¹¹ CONR³²¹R³³¹, —C₀₋₈alkyl-CONR³¹¹R³²¹, —C₀₋₈alkyl-CON(R³¹¹)S(O)₀₋₂R³²¹, —C₀₋₈alkyl-CO₂R³¹¹, —C₀₋₈alkyl-S(O)₀₋₂R³¹¹, —C₀₋₈alkyl-O—C₁₋₈alkyl, —C₀₋₈alkyl-O—C₀₋₈alkylC₃₋₈cycloalkyl, —C₀₋₈alkyl-O—C₀₋₈alkylheterocyclyl, —C₀₋₈alkyl-O—C₀₋₈alkylaryl, —C₀₋₈alkylaryl, —C₀₋₈alkylhetaryl, —C₀₋₈alkylheterocyclyl, —C₀₋₈alkyl-O—C₀₋₈alkylhetaryl, —C₀₋₈alkyl-S—C₀₋₈alkyl, —C₀₋₈alkyl-S—C₀₋₈alkylC₃₋₈cycloalkyl, —C₀₋₈alkyl-S—C₀₋₈alkylheterocyclyl, —C₀₋₈alkyl-S—C₀₋₈alkylaryl, —C₀₋₈alkyl-S—C₀₋₈alkylhetaryl, —C₀₋₈alkyl-N(R³¹¹)—C₀₋₈alkyl, —C₀₋₈alkyl-N(R³¹¹)—C₀₋₈alkylC₃₋₈cycloalkyl, —C₀₋₈alkyl-N(R³¹¹)—C₀₋₈alkylheterocyclyl, —C₀₋₈alkyl-N(R³¹¹)—C₀₋₈alkylaryl, —C₀₋₈alkyl-N(R³¹¹)—C₀₋₈alkylhetaryl, —C₀₋₈alkyl-NR³¹¹R³²¹, —C₂₋₈alkenyl, —C₂₋₈alkynyl, NO₂, CN, CF₃, OCF₃, OCHF₂; provided that Q¹ is not N-methyl-2-indolyl, N-(phenylsulfonyl)-2-indolyl, or N-tert-butoxycarbonyl

W is independently, hydrogen, —N(C₀₋₈alkyl)(C₀₋₈alkyl), hydroxyl, halogen, oxo, aryl (optionally substituted with 1 or more R³¹ groups), hetaryl (optionally substituted with 1 or more R³¹ groups), C₁₋₆ alkyl, —C₀₋₈alkylC₃₋₈cycloalkyl, —C₀₋₈alkyl-NR³¹²S(O)₀₋₂R³²², —C₀₋₈alkyl-NR³¹¹S(O)₀₋₂NR³²¹R³³¹, —C₀₋₈alkyl-NR³¹¹CO₂R, —C₀₋₈alkyl-CON(R³¹¹)S(O)₀₋₂R³²¹, —C₀₋₈alkyl-S(O)₀₋₂NR³¹²R³²², —C₀₋₈alkyl-NR³¹²COR³²², —C₀₋₈alkyl-NR³¹²CONR³²²R³³², —C₀₋₈alkyl-CONR³¹²R³²², —C₀₋₈alkyl-CO₂R³¹², —C₀₋₈alkylS(O)₀₋₂R³¹², —C₀₋₈alkyl-O—C₁₋₈alkyl, —C₀₋₈alkyl-O—C₀₋₈alkylcyclyl, —C₀₋₈alkyl-O—C₀₋₈alkylheterocycloalkyl, —C₀₋₈alkyl-O—C₀₋₈alkylaryl, —Oaryl, —C₀₋₈alkyl-O—C₀₋₈alkylhetaryl, —C₀₋₈alkylaryl, —C₀₋₈alkylhetaryl, —C₀₋₈alkylheterocyclyl, —C₀₋₈alkyl-S—C₀₋₈alkyl, —C₀₋₈alkyl-S—C₀₋₈alkylC₃₋₈cycloalkyl, —C₀₋₈alkyl-S—C₀₋₈alkylheterocycloalkyl, —C₀₋₈alkyl-S—C₀₋₈alkylaryl, —C₀₋₈alkyl-S—C₀₋₈alkylhetaryl, —C₀₋₈alkyl-N(R³¹²)—C₀₋₈alkyl, —C₀₋₈alkyl-N(R³¹²)—C₀₋₈alkylC₃₋₈cycloalkyl, —C₀₋₈alkyl-N(R³¹²)—C₀₋₈alkylheterocycloalkyl, —C₀₋₈alkyl-N(R³¹²)—C₀₋₈alkylaryl, —C₀₋₈alkyl-N(R³¹²)—C₀₋₈alkylhetaryl, —C₀₋₈alkyl-NR³¹²R³²², —C₂₋₈alkenyl, —C₂₋₈alkynyl, NO₂, CN, CF₃, OCF₃, OCHF₂; provided that Q¹ is not 4-benzyloxy-2-indolyl;

Y is independently, hydrogen, —N(C₀₋₈alkyl)(C₀₋₈alkyl), hydroxyl, halogen, oxo, aryl(optionally substituted with 1 or more R³¹ groups), hetaryl(optionally substituted with 1 or more R³¹ groups), C₀₋₆ alkyl, —C₀₋₈alkylC₃₋₈cycloalkyl, —C₀₋₈alkyl-NR³¹¹S(O)₀₋₂R³²¹, —C₀₋₈alkyl-NR³¹¹S(O)₀₋₂ NR³²¹R³³¹, —C₀₋₈alkyl-NR³¹¹CO₂R³²¹, —C₀₋₈alkyl-CON(R³¹¹)S(O)₀₋₂R³²¹, —C₀₋₈alkyl-S(O)₀₋₂NR³¹¹R³²¹, —C₀₋₈alkyl-NR³¹¹COR³²¹, —C₀₋₈alkyl-NR³¹¹CONR³²¹R³³¹, —C₀₋₈alkyl-CONR³¹¹R³²¹, —C₀₋₈alkyl-CO₂R³¹¹, —C₀₋₈alkylS(O)₀₋₂R³¹¹, —C₀₋₈alkyl-O—C₁₋₈alkyl, —C₀₋₈alkyl-O—C₀₋₈alkylC₃₋₈cycloalkyl, —C₀₋₈alkyl-O—C₀₋₈alkylheterocycloalkyl, —C₀₋₈alkyl-O—C₀₋₈alkylaryl, —C₀₋₈alkyl-O—C₀₋₈alkylhetaryl, —C₀₋₈alkylaryl, —C₀₋₈alkylhetaryl, —C₀₋₈alkylheterocyclyl, —C₀₋₈alkyl-S—C₀₋₈alkyl, —C₀₋₈alkyl-S—C₀₋₈alkylC₃₋₈cycloalkyl, —C₀₋₈alkyl-S—C₀₋₈alkylheterocycloalkyl, —C₀₋₈alkyl-S—C₀₋₈alkylaryl, —C₀₋₈alkyl-S—C₀₋₈alkylhetaryl, —C₀₋₈alkyl-N(R³¹¹)—C₀₋₈alkyl, —C₀₋₈alkyl-N(R³¹¹)—C₀₋₈alkylC₃₋₈cycloalkyl, —C₀₋₈alkyl-N(R³¹¹)—C₀₋₈alkylheterocycloalkyl, —C₀₋₈alkyl-N(R³¹¹)—C₀₋₈alkylaryl, —C₀₋₈alkyl-N(R³¹¹)—C₀₋₈alkylhetaryl, —C₀₋₈alkyl-NR³¹¹R³²¹, —C₂₋₈alkenyl, —C₂₋₈alkynyl, NO₂, CN, CF₃, OCF₃, OCHF₂; provided that Q¹ is not 2-carboxy-5-benzo[b]thiophenyl;

G¹¹ is halo, oxo, —CF₃, —OCF₃, —OR³¹², —NR³¹²R³²², —C(O)R³¹², —C(O)C₃₋₈cycloalkyl, —CO₂C₃₋₈cycloalkyl, —CO₂R³¹², —C(═O)NR³¹²R³²², —NO₂, —CN, —S(O)₀₋₂R³¹², —SO₂NR³¹²R³²², NR³¹²(C═O)R³²², NR³¹²C(═O)OR³²², NR³¹²C(═O)NR³²²R³³², NR³¹²S(O)₀₋₂R³²², —C(═S)OR³¹², —C(═O)SR³¹², —NR³¹²C(═NR³²²)NR³³²R³⁴¹, —NR³¹²C(═NR³²²)OR³³², —NR³¹²C(═NR³²²)SR³³², —OC(═O)OR³¹², —OC(═O)NR³¹²R³²², —OC(═O)SR³¹², —SC(═O)OR³¹², —SC(═O)NR³¹²R³²², —P(O)OR³¹²OR³²², C₁₋₁₀alkylidene, C₀₋₁₀alkyl, C₂₋₁₀alkenyl, C₂₋₁₀alkynyl, —C₁₋₁₀alkoxyC₁₋₁₀alkyl, —C₁₋₁₀ alkoxyC₂₋₁₀alkenyl, —C₁₋₁₀alkoxyC₂₋₁₀alkynyl, —C₁₋₁₀alkylthioC₁₋₁₀alkyl, —C₁₋₁₀alkylthioC₂₋₁₀alkenyl, —C₁₋₁₀alkylthioC₂₋₁₀alkynyl, cycloC₃₋₈alkyl, cycloC₃₋₈alkenyl, -cycloC₃₋₈alkylC₁₋₁₀alkyl, -cycloC₃₋₈alkenylC₁₋₁₀alkyl, -cycloC₃₋₈alkylC₂₋₁₀alkenyl, -cycloC₃₋₈alkenylC₂₋₁₀alkenyl, -cycloC₃₋₈alkylC₂₋₁₀alkynyl, -cycloC₃₋₈alkenylC₂₋₁₀alkynyl, -heterocyclyl-C₀₋₁₀alkyl, heterocyclyl-C₂₋₁₀alkenyl, or -heterocyclyl-C₂₋₁₀alkynyl, any of which is optionally substituted with one or more independent halo, oxo, —CF₃, —OCF₃, —OR³¹³, —NR³¹³R³²³, —C(O)R³¹³, —CO₂R³¹³, —C(═O)NR³¹³R³²³, —NO₂, —CN, —S(O)₀₋₂R³¹³, —SO₂NR³¹³R³²³, —NR³¹³C(═O)R³²³, —NR³¹³C(═O)OR³²³, —NR³¹³C(═O)NR³²³R³³³, —NR³¹³S(O)₀₋₂R³²³, —C(═S)OR³¹³, —C(═)SR³¹³, —NR³¹³C(═NR³²³)NR³³³R³⁴², —NR³¹³C(═NR³²³)OR³³³, —NR³¹³C(═NR³²³)SR³³³, —OC(═O)OR, —OC(═O)NR³¹³R³²³, —OC(═O)SR³¹³, —SC(═O)OR³¹³, —P(O)OR³¹³OR³²³, or —SC(═O)NR³¹³R³²³ substituents;

or G¹¹ is aryl-C₀₋₁₀alkyl, aryl-C₂₋₁₀alkenyl, aryl-C₂₋₁₀alkynyl, hetaryl-C₀₋₁₀alkyl, hetaryl-C₂₋₁₀alkenyl, or hetaryl-C₂₋₁₀alkynyl, where the attachment point is from either the left or right as written, where any of which is optionally substituted with one or more independent halo, —CF₃, —OCF₃, —OR³¹³, —NR³¹³R³²³, —C(O)R³¹³, —CO₂R³¹³, —C(═O)NR³¹³R³²³, —NO₂, —CN, —S(O)₀₋₂R³¹³, —SO₂ NR³¹³R³²³, —NR³¹³C(═O)R³²³, —NR³¹³C(═O)OR³²³, —NR³¹³C(═O)NR³²³R³³³, —NR³¹³S(O)₀₋₂R³²³, —C(═S)OR³¹³, —C(═O)SR³¹³, —NR³²³C(═NR³¹³)NR³³³R³⁴², —NR³¹³C(═NR³²³)OR³³³, —NR³¹³C(═NR³²³)SR³³³, —OC(═O)OR³¹³, —OC(═O)NR³¹³R³²³, —OC(═O)SR³¹³, —SC(═O)OR³¹³, —P(O)OR³¹³OR³²³, or —SC(═O)NR³¹³R³²³ substituents; provided that G¹¹ is not N—CH₂ CO₂ H when R³ is 4-piperidinyl;

R³¹, R³², R³³, R³¹¹, R³²¹, R³³¹, R³¹², R³²², R³³², R³⁴¹, R³¹³, R³²³, R³³³, and R³⁴², in each instance, is independently

C₀₋₈alkyl optionally substituted with an aryl, heterocyclyl or hetaryl substituent, or C₀₋₈alkyl optionally substituted with 1-6 independent halo, —CON(C₀₋₈alkyl)(C₀₋₈alkyl), —CO(C₀₋₈alkyl), —OC₀₋₈alkyl, -Oaryl, -Ohetaryl, -Oheterocyclyl, —S(O)₀₋₂aryl, —S(O)₀₋₂hetaryl, —S(O)₀₋₂heterocyclyl, —S(O)₀₋₂C₀₋₈alkyl, —N(C₀₋₈alkyl)(C₀₋₈alkyl), —N(C₀₋₈alkyl)CON(C₀₋₈alkyl)(C₀₋₈alkyl), —N(C₀₋₈alkyl)CO(C₁₋₈alkyl), —N(C₀₋₈alkyl)CO(C₃₋₈cycloalkyl), —N(C₀₋₈alkyl)CO₂(C₁₋₈alkyl), —S(O)₁₋₂N(C₀₋₈alkyl)(C₀₋₈alkyl), —NR¹¹S(O)₁₋₂(C₀₋₈alkyl), —CON(C₃₋₈cycloalkyl)(C₃₋₈cycloalkyl), —CON(C₀₋₈alkyl)(C₃₋₈cycloalkyl), —N(C₃₋₈cycloalkyl)CON(C₀₋₈alkyl)(C₀₋₈alkyl), —N(C₃₋₈cycloalkyl)CON(C₃₋₈cycloalkyl)(C₀₋₈alkyl), —N(C₀₋₈alkyl)CON(C₃₋₈cycloalkyl)(C₀₋₈alkyl), —N(C₀₋₈alkyl)CO₂(C₃₋₈cycloalkyl), —N(C₃₋₈cycloalkyl)CO₂(C₃₋₈cycloalkyl), S(O)₁₋₂N(C₀₋₈alkyl)(C₃₋₈cycloalkyl), —NR¹¹S(O)₁₋₂(C₃₋₈cycloalkyl), C₂₋₈alkenyl, C₂₋₈alkynyl, CN, CF₃, OH, or optionally substituted aryl substituents; such that each of the above aryl, heterocyclyl, hetaryl, alkyl or cycloalkyl groups may be optionally, independently substituted with —N(C₀₋₈alkyl)(C₀₋₈alkyl), hydroxyl, halogen, oxo, aryl, hetaryl, C₀₋₆alkyl, C₀₋₈alkylcyclyl, —C₀₋₈alkyl-N(C₀₋₈alkyl)-S(O)₀₋₂-(C₀₋₈alkyl), —C₀₋₈alkyl-S(O)₀₋₂—N(C₀₋₈alkyl)(C₀₋₈alkyl), —C₀₋₈alkyl-N(C₀₋₈alkyl)CO(C₀₋₈alkyl), —C₀₋₈alkyl-N(C₀₋₈alkyl)CO—N(C₀₋₈alkyl)(C₀₋₈alkyl), —C₀₋₈alkyl-CO—N(C₀₋₈alkyl)(C₀₋₈alkyl), —C₁₋₈alkyl-CO₂—(C₀₋₈alkyl), —C₀₋₈alkylS(O)₀₋₂-(C₀₋₈alkyl), —C₀₋₈alkyl-O—C₁₋₈alkyl, —C₀₋₈alkyl-O—C₀₋₈alkylcyclyl, —C₀₋₈alkyl-O—C₀₋₈alkylheterocyclyl, —C₀₋₈alkyl-O—C₀₋₈alkylaryl, —C₀₋₈alkyl-O—C₀₋₈alkylhetaryl, —C₀₋₈alkyl-S—C₀₋₈alkyl, —C₀₋₈alkyl-S—C₀₋₈alkylcyclyl, —C₀₋₈alkyl-S—C₀₋₈alkylheterocyclyl, —C₀₋₈alkyl-S—C₀₋₈alkylaryl, —C₀₋₈alkyl-S—C₀₋₈alkylhetaryl, —C₀₋₈alkyl-N(C₀₋₈alkyl)-C₀₋₈alkyl, —C₀₋₈alkyl-N(C₀₋₈alkyl)-C₀₋₈alkylcyclyl, —C₀₋₈alkyl-N(C₀₋₈alkyl)-C₀₋₈alkylheterocyclyl, —C₀₋₈alkyl-N(C₀₋₈alkyl)-C₀₋₈alkylaryl, —C₀₋₈alkyl-N(C₀₋₈alkyl)-C₀₋₈alkylhetaryl, C₂₋₈alkenyl, C₂₋₈alkynyl, NO₂, CN, CF₃, OCF₃, OCHF₂,

—C₀₋₈alkyl-C₃₋₈cycloalkyl,

—C₀₋₈alkyl-O—C₀₋₈alkyl,

—C₀₋₈alkyl-N(C₀₋₈alkyl)(C₀₋₈alkyl),

—C₀₋₈alkyl-S(O)₀₋₂—C₀₋₈alkyl, or

heterocyclyl optionally substituted with 1-4 independent C₀₋₈alkyl, cyclyl, or substituted cyclyl substituents;

E¹ in each instance is independently halo, —CF₃, —OCF₃, —OR², —NR³¹R³², —C(═O)R³¹, —CO₂R³¹, —CONR³¹R³², —NO₂, —CN, —S(O)₀₋₂R³¹, —S(O)₀₋₂NR³¹R³², —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³¹, —NR³¹C(═NR³²)OR³³, —NR³¹C(═NR³¹)SR³¹, —OC(═O)OR³¹, —OC(═O)NR³¹R³², —OC(═O)SR³¹, —SC(═O)OR³¹, —SC(═O)NR³¹R³², C₀₋₁₀alkyl, C₂₋₁₀alkenyl, C₂₋₁₀alkynyl, —C₁₋₁₀alkoxyC₁₋₁₀alkyl, —C₁₋₁₀alkoxyC₂₋₁₀alkenyl, —C₁₋₁₀alkoxyC₂₋₁₀alkynyl, —C₁₋₁₀alkylthioC₁₋₁₀alkyl, —C₁₋₁₀alkylthioC₂₋₁₀alkenyl, —C₁₋₁₀alkylthioC₂₋₁₀alkynyl, cycloC₃₋₈alkyl, cycloC₃₋₈alkenyl, -cycloC₃₋₈alkylC₁₋₁₀alkyl, -cycloC₃₋₈alkenylC₁₋₁₀alkyl, -cycloC₃₋₈alkylC₂₋₁₀alkenyl, -cycloC₃₋₈alkenylC₂₋₁₀alkenyl, -cycloC₃₋₈alkylC₂₋₁₀alkynyl, -cycloC₃₋₈alkenylC₂₋₁₀alkynyl, -heterocyclyl-C₀₋₁₀alkyl, -heterocyclyl-C₂₋₁₀alkenyl, or -heterocyclyl-C₂₋₁₀alkynyl, any of which is optionally substituted with one or more independent halo, oxo, —CF₃, —OCF₃, —OR³¹, —NR³¹R³², —C(═O)R³¹, —CO₂R³¹, —C(═O)NR³¹R³², —NO₂, —CN, —S(═O)₀₋₂R³¹, —SO₂NR³¹, —NR³¹C(═O)R³², —NR³¹C(═O)OR³¹, —NR³¹C(═O)NR³²R³³, —NR³¹S(O)₀₋₂R³¹, —C(═S)OR³¹, —C(═O)SR³¹, —NR³¹C(═NR³²)NR³³R³¹, —NR³¹C(═NR³²)OR³³, —NR³¹C(═NR³²)SR³³, —OC(═O)OR³¹, —OC(═O)NR³¹³², —OC(═O)SR³¹, —SC(═O)OR³¹, or —SC(═O)NR³¹R³² substituents;

or E¹ in each instance is independently aryl-C₀₋₁₀alkyl, aryl-C₂₋₁₀alkenyl, aryl-C₂₋₁₀alkynyl, hetaryl-C₀₋₁₀alkyl, hetaryl-C₂₋₁₀alkenyl, or hetaryl-C₂₋₁₀alkynyl, where the attachment point is from either the left or right as written, where any of which is optionally substituted with one or more independent halo, —CF₃, —OCF₃, —OR³¹, —NR³¹R³², —C(O)R³¹, —CO₂R³¹, —C(═O)NR³¹R³², —NO₂, —CN, —S(O)₀₋₂R³¹, —S(O)₀₋₂NR³¹R³², —NR³¹C(═O)R³², —NR³¹C(═O)OR³², —NR³¹C(═O)NR³²R³³, —NR³¹S(O)₀₋₂R³², —C(═S)OR³¹, —C(═O)SR³¹, —NR³¹C(═NR³²)NR³³R³¹, —NR³¹C(═NR³²)OR³³, —NR³¹C(═NR³²)SR³³, —OC(═O)OR³¹, —OC(═O)NR³¹R³², —OC(═O)SR³¹, —SC(═O)OR³¹, or —SC(═O)NR³¹R³² substituents;

in the cases of —NR³¹R³², —NR³¹¹R³²¹, —NR³¹²R³²², —NR³³²R³⁴¹, —NR³¹³R³²³, and —NR³²³R³³³, the respective R³¹ and R³², R³¹¹ and R³²¹, R³¹² and R³²², R³³¹ and R³⁴¹, R³¹³ and R³²³, and R³²³ and R³³³ are optionally taken together with the nitrogen atom to which they are attached to form a 3-10 membered saturated or unsaturated ring; wherein said ring in each instance independently is optionally substituted by one or more independent —N(C₀₋₈alkyl)(C₀₋₈alkyl), hydroxyl, halogen, oxo, aryl, hetaryl, C₀₋₆alkyl, —C₀₋₈alkylC₃₋₈cycloalkyl, —C₀₋₈alkyl-N(C₀₋₈alkyl)S(O)₀₋₂C₀₋₈alkyl, —C₀₋₈alkyl-N(C₀₋₈alkyl)S(O)₀₋₂N(C₀₋₈alkyl)(C₀₋₈alkyl), —C₀₋₈alkyl-N(C₀₋₈alkyl)CO₂(C₀₋₈alkyl), —C₀₋₈alkyl-CON((C₀₋₈alkyl))S(O)₀₋₂(C₀₋₈alkyl), —C₀₋₈alkyl-S(O)₀₋₂N(C₀₋₈alkyl)(C₀₋₈alkyl), —C₀₋₈alkyl-N(C₀₋₈alkyl)CO(C₀₋₈alkyl), —C₀₋₈alkyl-N(C₀₋₈alkyl)CON(C₀₋₈alkyl)(C₀₋₈alkyl), —C₀₋₈alkyl-CON(C₀₋₈alkyl)(C₀₋₈alkyl), —C₀₋₈alkyl-CO₂ (C₀₋₈alkyl), —C₀₋₈alkylS(O)₀₋₂(C₀₋₈alkyl), —C₀₋₈alkyl-O—C₀₋₈alkyl, —C₀₋₈alkyl-O—C₀₋₈alkylcyclyl, —C₀₋₈alkyl-O—C₀₋₈alkylheterocycloalkyl, —C₀₋₈alkyl-O—C₀₋₈alkylaryl, -Oaryl, —C₀₋₈alkyl-O—C₀₋₈alkylhetaryl, —C₀₋₈alkyl-S—C₀₋₈alkyl, —C₀₋₈alkyl-S—C₀₋₈alkylC₃₋₈cycloalkyl, —C₀₋₈alkyl-S—C₀₋₈alkylheterocycloalkyl, —C₀₋₈alkyl-S—C₀₋₈alkylaryl, —C₀₋₈alkyl-S—C₀₋₈alkylhetaryl, —C₀₋₈alkyl-N(C₀₋₈alkyl)-C₀₋₈alkyl, —C₀₋₈alkyl-N(C₀₋₈alkyl)-C₀₋₈alkylC₃₋₈cycloalkyl, —C₀₋₈alkyl-N(C₀₋₈alkyl)-C₀₋₈alkylheterocycloalkyl, —C₀₋₈alkyl-N(C₀₋₈alkyl)-C₀₋₈alkylaryl, —C₀₋₈alkyl-N(C₀₋₈alkyl)-C₀₋₈alkylhetaryl, —C₀₋₈alkyl-N(C₀₋₈alkyl)(C₀₋₈alkyl), C₂₋₈alkenyl, C₂₋₈alkynyl, NO₂, CN, CF₃, OCF₃, or OCHF₂ substituents; wherein said ring in each instance independently optionally includes one or more heteroatoms other than the nitrogen;

m is 0, 1, 2, or 3; n is 0, 1, 2, 3, or 4; and aa is 0 or 1.

In some aspects, the mTOR pathway is activated in the tumor. In some aspects, the mTOR inhibitor is a dual mTORC1 and mTORC2 inhibitor.

In some aspects, the mTOR inhibitor is a compound of (1r,4r)-4-(4-amino-5-(7-methoxy-1H-indol-2-yl)imidazo[1,5-f][1,2,4]triazin-7-yl)cyclohexanecarboxylic acid (OSI-027) or a pharmaceutically acceptable salt thereof, which has the formula:

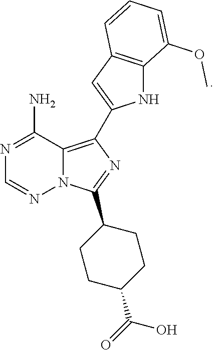

In some aspects, the mTOR inhibitor is a tromethamine salt of OSI-027.

### Angiogenesis Inhibitor

In some aspects, the angiogenesis inhibitor can be any angiogenesis inhibitor, including any such inhibitor approved for marketing by a government regulatory authority.

In some aspects, the angiogenesis inhibitor can be any agent capable of decreasing the activity of aFGF, bFGF, TGF-α, TGF-β, HGF, TNF-α, angiogenin, or IL-F, or other positive regulator of angiogenesis.

In some aspects, the angiogenesis inhibitor can be an endothelial cell growth inhibitor, extracellular matrix breakdown inhibitor, or angiogenesis signaling cascade inhibitor such as a VEGF activity inhibitor.

In some aspects, the angiogenesis inhibitor can be a VEGF inhibitor or a VEGFR inhibitor and can be an antibody or small molecule.

Non-limiting examples of angiogenesis inhibitors include Anti-VEGFR2 antibodies or KDR antibodies, such as IMC-1121b (ImClone Systems) or CDP-791 (Celltech/UCB/ImClone Systems), VEGFR inhibitors, such as SU-5416 and SU-6668 (Sugen Inc. of South San Francisco, Calif., USA), or as described in, for example International Application Nos. WO 99/24440, WO 99/62890, WO 95/21613, WO 99/61422, WO 98/50356, WO 99/10349, WO 97/32856, WO 97/22596, WO 98/54093, WO 98/02438, WO 99/16755, and WO 98/02437, and U.S. Pat. Nos. 5,883,113, 5,886,020, 5,792,783, 5,834,504 and 6,235,764; VEGF inhibitors such as IM862 (Cytran Inc. of Kirkland, Wash., USA); angiozyme, a synthetic ribozyme from Ribozyme (Boulder, Colo.) and Chiron (Emeryville, Calif.); and antibodies to VEGF, such as bevacizumab (e.g. AVASTIN®, Genentech, South San Francisco, Calif.), a recombinant humanized antibody to VEGF; integrin receptor antagonists and integrin antagonists, such as to αᵥβ₃, αᵥβ₅ and αᵥβ₆ integrins, and subtypes thereof, e.g. cilengitide (EMD 121974), or the anti-integrin antibodies, such as for example αᵥβ₃ specific humanized antibodies (e.g. VITAXIN®); factors such as IFN-alpha (U.S. Pat. Nos. 41,530,901, 4,503,035, and 5,231,176); angiostatin and plasminogen fragments (e.g. kringle 1-4, kringle 5, kringle 1-3 (O'Reilly, M. S. et al. (1994) Cell 79:315-328; Cao et al. (1996) J. Biol. Chem. 271: 29461-29467; Cao et al. (1997) J. Biol. Chem. 272:22924-22928); endostatin (O'Reilly, M. S. et al. (1997) Cell 88:277; and International Patent Publication No. WO 97/15666); thrombospondin (TSP-1; Frazier, (1991) Curr. Opin. Cell Biol. 3:792); platelet factor 4 (PF4); plasminogen activator/urokinase inhibitors; urokinase receptor antagonists; heparinases; fumagillin analogs such as TNP-4701; suramin and suramin analogs; angiostatic steroids; bFGF antagonists; flk-1 and flt-1 antagonists; anti-angiogenesis agents such as MMP-2 (matrix-metalloproteinase 2) inhibitors and MMP-9 (matrix-metalloproteinase 9) inhibitors. Examples of useful matrix metalloproteinase inhibitors are described in International Patent Publication Nos. WO 96/33172, WO 96/27583, WO 98/07697, WO 98/03516, WO 98/34918, WO 98/34915, WO 98/33768, WO 98/30566, WO 90/05719, WO 99/52910, WO 99/52889, WO 99/29667, and WO 99/07675, European Patent Publication Nos. 818,442, 780,386, 1,004,578, 606,046, and 931,788; Great Britain Patent Publication No. 9912961, and U.S. Pat. Nos. 5,863,949 and 5,861,510. Preferred MMP-2 and MMP-9 inhibitors are those that have little or no activity inhibiting MMP-1. More preferred, are those that selectively inhibit MMP-2 and/or MMP-9 relative to the other matrix-metalloproteinases (i.e. MMP-1, MMP-3, MMP-4, MMP-5, MMP-6, MMP-7, MMP-8, MMP-10, MMP-11, MMP-12, and MMP-13).

Other angiogenesis inhibitors include bevacizumab (Avastin®), aflibercept, sunitinib (Sutent®), sorafenib (Nexavar®), vatalanib, AMG-706, CP-547632, pazopanib, ABT-869, IMC-1C11, cediranib and OSI-930. More preferred angiogenesis inhibitors are bevacizumab (Avastin®) and sunitinib (Sutent®).

### Settings

In some aspects, the patient to be treated is refractory to treatment with the angiogenesis inhibitor as a single agent. Thus, for example, in one embodiment, the present invention provides a method for treating tumors or tumor metastases in a patient wherein the cells of the tumor or tumor metastasis are relatively insensitive or refractory to treatment with the angiogenesis inhibitor as a single agent, comprising administering to said patient simultaneously or sequentially a therapeutically effective amount of a combination of an mTOR inhibitor and an angiogenesis inhibitor. It will be appreciated by one of skill in the medical arts that there are many reasons why a patient may be refractory to treatment with the angiogenesis inhibitor as a single agent, one of which is that the tumor cells of the patient are relatively insensitive to inhibition by the tested angiogenesis inhibitor. It is also possible that a patient may be refractory to treatment with one type of angiogenesis inhibitor, but be sensitive to treatment with another type of angiogenesis inhibitor.

In some aspects, the patient is a human in need of treatment for cancer, a precancerous condition or lesion, or other forms of abnormal cell growth. The cancer may be, for example: non-small cell lung (NSCL) cancer, breast cancer, colon cancer, pancreatic cancer, lung cancer, bronchioloalveolar cell lung cancer, bone cancer, skin cancer, cancer of the head or neck, cutaneous or intraocular melanoma, uterine cancer, ovarian cancer, rectal cancer, cancer of the anal region, stomach cancer, gastric cancer, uterine cancer, carcinoma of the fallopian tubes, carcinoma of the endometrium, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the ureter, cancer of the kidney, renal cell carcinoma, carcinoma of the renal pelvis, mesothelioma, hepatocellular cancer, biliary cancer, chronic or acute leukemia, lymphocytic lymphomas, neoplasms of the central nervous system (CNS), spinal axis tumors, brain stem glioma, glioblastoma multiforme, astrocytomas, schwannomas, ependymomas, medulloblastomas, meningiomas, squamous cell carcinomas, pituitary adenomas, including refractory versions of any of the above cancers, or a combination of one or more of the above cancers. The precancerous condition or lesion includes, for example, the group consisting of oral leukoplakia, actinic keratosis (solar keratosis), precancerous polyps of the colon or rectum, gastric epithelial dysplasia, adenomatous dysplasia, hereditary nonpolyposis colon cancer syndrome (HNPCC), Barrett's esophagus, bladder dysplasia, and precancerous cervical conditions. Preferred embodiments of the cancer/tumor comprise ovarian, prostate, non small cell lung cancer, renal cell carcinoma, endometrial, glioblastoma, lymphoma or pancreatic cancer.

In some aspects, the mTOR inhibitor is introduced following occurrence of resistance to initial treatment with an angiogenesis inhibitor without an mTOR inhibitor.

In some aspects, the treatments result in tumor size reduction of 5%, 10%, 20%, 30%, 40%, 50%, 60%, 70%, or greater according to Response Evaluation Criteria In Solid Tumors (RECIST).

### Pharmaceutical Compositions

In some aspects, the present invention provides a pharmaceutical composition comprising an optional pharmaceutically acceptable carrier and/or excipient and, as active ingredient, a mTOR inhibitor and an angiogenesis inhibitor, or a pharmaceutically acceptable salt thereof, and optionally one or more other anti-cancer agents.

The above-described pharmaceutical compositions include compositions suitable for oral, rectal, topical, and parenteral (including subcutaneous, intramuscular, and intravenous) administration, although the most suitable route in any given case will depend on the particular host and nature and severity of the conditions for which the active ingredient is being administered. The pharmaceutical compositions may be conveniently presented in unit dosage form and prepared by any of the methods well known in the art of pharmacy.

The active ingredients of the pharmaceutical compositions can be combined in intimate admixture with a pharmaceutical carrier according to conventional pharmaceutical compounding techniques. The carrier may take a wide variety of forms depending on the form of preparation desired for administration, e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion, or as a water-in-oil liquid emulsion. In addition to the common dosage forms set out above, the active ingredients of the composition, or a pharmaceutically acceptable salt thereof, may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.05 mg to about 5 g of the active ingredient and each cachet or capsule preferably containing from about 0.05 mg to about 5 g of the active ingredient.

A formulation intended for the oral administration to humans may contain from about 0.5 mg to about 5 g of active agent, compounded with an appropriate and convenient amount of carrier material which may vary from about 5 to about 95 percent of the total composition. Unit dosage forms will generally contain between from about 1 mg to about 2 g of the active ingredient, typically 25 mg, 50 mg, 100 mg, 200 mg, 300 mg, 400 mg, 500 mg, 600 mg, 800 mg, or 1000 mg.

Compounds of the invention can be provided for formulation at high purity, for example at least about 90%, 95%, or 98% pure by weight.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, or the like. Further, the compositions can be in a form suitable for use in transdermal devices. These formulations may be prepared via conventional processing methods. As an example, a cream or ointment is prepared by admixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in molds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions may also be prepared in powder or liquid concentrate form.

### Administration

In some aspects, the mTOR inhibitor and the angiogenesis inhibitor are co-administered to the patient in the same formulation. In some aspects, mTOR inhibitor and the angiogenesis inhibitor are co-administered to the patient in different or separate formulations. In some aspects the administration of the mTOR inhibitor and the angiogenesis inhibitor to the patient is simultaneous. In some aspects the administration of the mTOR inhibitor and the angiogenesis inhibitor to the patient is sequential.

In conducting the treatment method of the present invention, the mTOR inhibitor and the angiogenesis inhibitor can be administered in any effective manner known in the art, such as by oral, topical, intravenous, intra-peritoneal, intramuscular, intra-articular, subcutaneous, intranasal, intra-ocular, vaginal, rectal, or intradermal routes, depending upon the type of cancer being treated, the type of the mTOR inhibitor and the angiogenesis inhibitor being used (for example, small molecule, antibody, RNAi, ribozyme or antisense construct), and the medical judgment of the prescribing physician as based, e.g., on the results of published clinical studies.

The mTOR inhibitor and the angiogenesis inhibitor can be administered either separately or together by the same or different routes, and in a wide variety of different dosage forms. For example, the mTOR inhibitor is preferably administered orally or parenterally. The angiogenesis inhibitor is preferably administered orally or parenterally. Where the mTOR inhibitor is OSI-027, oral administration is preferable. Where the angiogenesis inhibitor is bevacizumab, parenteral administration is preferable. Where the angiogenesis inhibitor is sunitinib, oral administration is preferable. Both the mTOR inhibitor and the angiogenesis inhibitor can be administered in single or multiple doses.

In some aspects, the mTOR inhibitor and the angiogenesis inhibitor are co-administered to the patient by the same route. In some aspects, the mTOR inhibitor and the angiogenesis inhibitor are co-administered to the patient by different routes.

The mTOR inhibitor and the angiogenesis inhibitor are typically administered to the patient in a dose regimen that provides for the most effective treatment of the cancer (from both efficacy and safety perspectives) for which the patient is being treated, as known in the art, and as disclosed below.

The amount of the mTOR inhibitor and the angiogenesis inhibitor administered and the timing of the administration will depend on the type (species, gender, age, weight, etc.) and condition of the patient being treated, the severity of the disease or condition being treated, and on the route of administration. For example, the mTOR inhibitor and the angiogenesis inhibitor can each be administered to a patient in doses ranging from about 0.001 to about 100 mg/kg or 0.01 to about 10 mg/kg of body weight per day in single or divided doses.

When the mTOR inhibitor is OSI-027, it can be administered in an amount of about 0.1 to about 100 mg/kg body weight on days of administration. Or, it can be administered in an amount of about 5 to about 65 mg/kg, or about 0.5 to about 5 mg/kg body weight on days of administration.

When the angiogenesis inhibitor is bevacizumab, it is preferably administered in an amount of about 1 to about 100 mg/kg body weight on days of administration, or approximately the label dosing.

When the angiogenesis inhibitor is sunitinib, it is preferably administered in an amount of about 10 to about 100 mg/kg body weight on days of administration, or approximately the label dosing.

For the above-described methods, OSI-027 and bevacizumab can be administered in a mass ratio of about 3 to about 50. Preferably, OSI-027 and bevacizumab are administered in a mass ratio of about 6 to about 15.

For the above-described methods, OSI-027 and sunitinib can be administered in a mass ratio of about 0.1 to about 3. Preferably, OSI-027 and sunitinib are administered in a mass ratio of about 0.2 to about 2.

In some aspects, the mTOR inhibitor and the angiogenesis inhibitor are administered on different days. In another embodiment, neither the mTOR inhibitor nor the angiogenesis inhibitor is administered on certain days.

The present invention further provides a method for treating tumors or tumor metastases in a patient, comprising administering to the patient simultaneously or sequentially a therapeutically effective amount of a combination of an mTOR inhibitor and an angiogenesis inhibitor that produce synergistic anti-tumor effects, with one or more other anti-cancer agents. The anti-cancer agents include, for example: alkylating agents or agents with an alkylating action, such as cyclophosphamide (CTX; e.g. CYTOXAN®), chlorambucil (CHL; e.g. LEUKERAN®), cisplatin (CisP; e.g. PLATINOL®) busulfan (e.g. MYLERAN®), melphalan, carmustine (BCNU), streptozotocin, triethylenemelamine (TEM), mitomycin C, and the like; anti-metabolites, such as methotrexate (MTX), etoposide (VP16; e.g. VEPESID®), 6-mercaptopurine (6 MP), 6-thiocguanine (6TG), cytarabine (Ara-C), 5-fluorouracil (5-FU), capecitabine (e.g. XELODA®), dacarbazine (DTIC), and the like; antibiotics, such as actinomycin D, doxorubicin (DXR; e.g. ADRIAMYCIN®), daunorubicin (daunomycin), bleomycin, mithramycin and the like; alkaloids, such as vinca alkaloids such as vincristine (VCR), vinblastine, and the like; and other antitumor agents, such as paclitaxel (e.g. TAXOL®) and paclitaxel derivatives, the cytostatic agents, glucocorticoids such as dexamethasone (DEX; e.g. DECADRON®) and corticosteroids such as prednisone, nucleoside enzyme inhibitors such as hydroxyurea, amino acid depleting enzymes such as asparaginase, leucovorin and other folic acid derivatives, and similar, diverse antitumor agents. The following agents may also be used as additional agents: arnifostine (e.g. ETHYOL®), dactinomycin, mechlorethamine (nitrogen mustard), streptozocin, cyclophosphamide, lomustine (CCNU), doxorubicin lipo (e.g. DOXIL®), gemcitabine (e.g. GEMZAR®), daunorubicin lipo (e.g. DAUNOXOME®), procarbazine, mitomycin, docetaxel (e.g. TAXOTERE®), aldesleukin, carboplatin, oxaliplatin, cladribine, camptothecin, CPT 11 (irinotecan), 10-hydroxy 7-ethyl-camptothecin (SN38), floxuridine, fludarabine, ifosfamide, idarubicin, mesna, interferon beta, interferon alpha, mitoxantrone, topotecan, leuprolide, megestrol, melphalan, mercaptopurine, plicamycin, mitotane, pegaspargase, pentostatin, pipobroman, plicamycin, tamoxifen, teniposide, testolactone, thioguanine, thiotepa, uracil mustard, vinorelbine, chlorambucil. For the above-described methods, the progression-free survival time (PFST) for patients treated in accordance with said method of this invention is at least about 4, 8, 16, 32, 64 or 128 weeks from initiation of the therapy of this invention; preferably the PFST is in the range of about 16 to about 64 weeks from initiation of the therapy of this invention.

The increase in the progression-free survival time expected for patients treated in accordance with the methods of this invention is greater than about 8 weeks to about 1.5 years compared to control (observation based no clinical by the skilled practitioners).

This invention will be better understood from the Experimental Details that follow. However, one skilled in the art will readily appreciate that the specific methods and results discussed are merely illustrative of the invention as described more fully in the claims which follow thereafter, and are not to be considered in any way limited thereto.

### EXAMPLES

Growth inhibition of tumor cell lines by combining an mTOR inhibitor with an angiogenesis inhibitor is shown. Specifically, five pharmacological studies (Nos. 1-5) with OSI-027 and angiogenesis inhibitors bevacizumab and sunitinib are illustrated below. The in vivo results from these studies are intended to illustrate the present invention without posing any limitation to it.

### Materials and Methods

With respect to study Nos. 1 and 2, male athymic nude nu/nu mice (6-8 wks, 25-32 g, Harlan) were allowed to acclimate for a minimum of one week prior to initiation of the study. To evaluate tumor growth inhibition, DU145 tumor fragments were implanted s.c. in the right flank of nude mice. Tumors were established to 200±50 mm³ in size before randomization into treatment groups of 8 mice each.

Regarding study Nos. 3 and 4, female athymic nude nu/nu CD-1 mice (6-8 wks, 20-29 g, Charles River Laboratories, Wilmington, Mass., USA) were allowed to acclimate for a minimum of one week prior to initiation of the study. To evaluate tumor growth inhibition, cells were harvested from cell culture flasks during exponential cell growth, washed twice with sterile PBS, counted and resuspended in PBS to a suitable concentration before s.c. implantation on the right flank of nu/nu CD-1 mice. Tumors were established to 200±50 mm³ in size before randomization into treatment groups of 8 mice each.

With respect to study No. 5, female athymic nude nu/nu mice (11-12 wks, 20-27 g, Harlan) were allowed to acclimate for a minimum of one week prior to initiation of the study. To evaluate tumor growth inhibition, SKOV3 tumor fragments were implanted s.c. in the right flank of nude mice. Tumors were established to mean tumor volume per treatment group of 84.6 mm³-85.3 mm³ of 8 mice each.

All of the Tumor Growth Inhibition (TGI) studies were performed using 20% Trappsol as the vehicle with once-daily oral administration of OSI-027 for 14 or 28 consecutive days. Body weights were determined twice weekly along with tumor volume (V=[length×(width)]/2) measurements using Vernier calipers. Tumor growth inhibition was determined at different time points by the following formula:

$$\% TGI = ( \frac{1 - [ \frac{{T}_{t} / {T}_{0}}{{C}_{t} / {C}_{0}} ]}{1 - [ {C}_{0} / {C}_{t} ]} ) \times 100$$

where Tₜ=median tumor volume of treated at time t; T₀=median tumor volume of treated at time 0; Cₜ=median tumor volume of control at time t; and C₀=median tumor volume of control at time 0. The average TGI over the dosing period was then calculated and reported.

Tumor regressions were determined and calculated as follows using the formula: % Regression=100(T₀−Tᵢ)/T₀; where T₀ is the mean tumor volume for treated group at the initiation of treatment and Tᵢ is the mean tumor volume for that group at time x.

### Results

Study No. 1: In vivo efficacy study of OSI-027 or bevacizumab (Avastin®) alone and in combination of OSI-027 with bevacizumab (Avastin®) in DU145 prostate carcinoma xenografts.

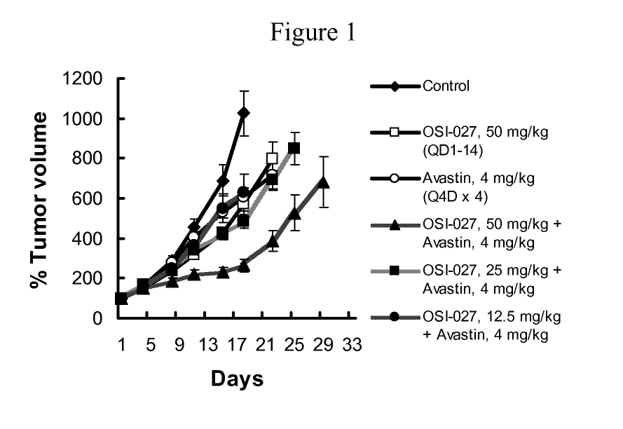

As shown in below Table 1 and FIG. 1 below, daily oral administration of OSI-027 at 50 mg/kg for 14 days resulted in only 21% mean tumor growth inhibition (TGI) as measured during the dosing period. Bevacizumab (Avastin) as a single agent administered at 4 mg/kg q4d×4 resulted in only 16% mean tumor growth inhibition (TGI) as measured during the dosing period. In the combination groups, all mice received daily oral administration OSI-027 at 12.5, 25 or 50 mg/kg every day with bevacizumab at 4 mg/kg q4d×4 intravenously. The combination of bevacizumab with OSI-027 at 50 mg/kg resulted in a mean TGI of 51%. The combination was well tolerated with an average 8% body weight loss (BWL). Therefore, OSI-027 in combination with bevacizumab demonstrated therapeutic synergy with improved tumor growth inhibition compared with treatment by single agent alone.

TABLE 1

| | Avg. | % | | Morbidity |
|---|---|---|---|---|
| Drug | TGI | Reg. | BWL | (Mortality) |
| OSI-027, 50 mg/kg qd | 21% | 0% | 6% | 0/8 |
| Avastin, 4 mg/kg q4d | 16% | 0% | 4% | 0/8 |
| Avastin + OSI-027, 12.5 mg/kg qd | 17% | 0% | 4% | 0/8 |
| Avastin + OSI-027, 25 mg/kg qd | 22% | 0% | 4% | 0/8 |
| Avastin + OSI-027, 50 mg/kg qd | 51% | 0% | 8% | 0/8 |

Study No. 2: In vivo efficacy study of OSI-027 or bevacizumab (Avastin ®) alone and in combination of OSI-027 with bevacizumab (Avastin ®) in DU145 prostate carcinoma xenografts with extended dosing for 28 days.

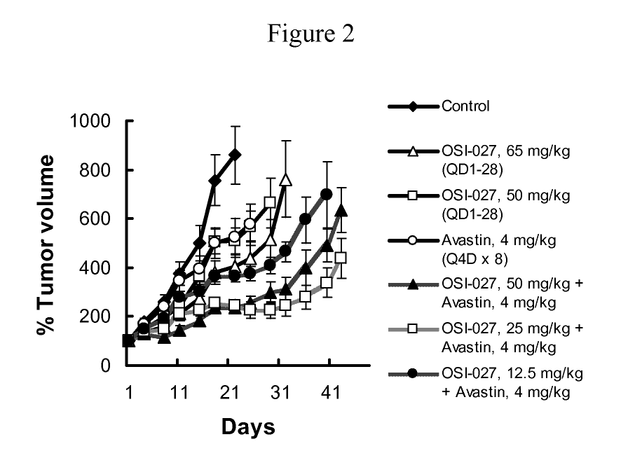

As shown in below Table 2 and FIG. 2 below, daily oral administration of OSI-027 at 50 and 65 mg/kg for 28 days resulted in 40% and 58% mean tumor growth inhibition (TGI) as measured during the dosing period, respectively. Bevacizumab (Avastin) as a single agent administered at 4 mg/kg q4d×8 demonstrated 32% TGI. In the combination groups, all mice received daily oral administration OSI-027 at 12.5, 25 or 50 mg/kg every day with bevacizumab at 4 mg/kg q4d×8 intravenously. The combination of bevacizumab with OSI-027 at 50 mg/kg resulted in a mean TGI of 82%, and combinations of bevacizumab with OSI-027 at 25 mg/kg demonstrated 62% TGI respectively. Therefore, OSI-027 in combination with bevacizumab demonstrated therapeutic synergy with improved tumor growth inhibition compared with treatment by single agent alone.

TABLE 2

| | Avg. | % | | Morbidity |
|---|---|---|---|---|
| Drug | TGI | Reg. | BWL | (Mortality) |
| OSI-027, 50 mg/kg qd | 40% | 0% | 14% | 0/8 |
| OSI-027, 65 mg/kg qd | 58% | 0% | 17% | 0/8 |
| Avastin, 4 mg/kg q4d | 32% | 0% | 9% | 0/8 |
| Avastin + OSI-027, 12.5 mg/kg qd | 44% | 0% | 3% | 0/8 |
| Avastin + OSI-027, 25 mg/kg qd | 62% | 0% | 3% | 0/8 |
| Avastin + OSI-027, 50 mg/kg qd | 82% | 0% | 16% | 0/8 |

Study No.3: In vivo efficacy study of OSI-027 or sunitinib (Sutent ®) alone and the combination of OSI-027 with sunitinib (Sutent ®) in H292 lung carcinoma xenografts

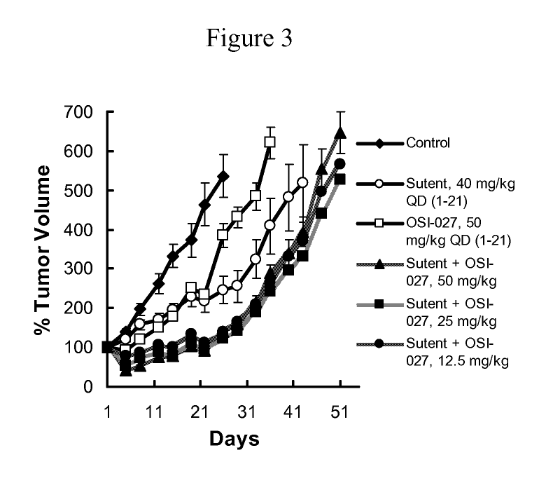

As shown in Table 3 and FIG. 3 below, daily oral administration of OSI-027 at 50 mg/kg for 21 days resulted in significant (83%) mean tumor growth inhibition (TGI) as measured during the dosing period with 8% tumor regression. Sunitinib (Sutent) as a single agent administered daily at 40 mg/kg for 21 days resulted in 55% TGI. In the combination groups, all mice received daily oral administration of OSI-027 at 12.5, 25 or 50 mg/kg every day with sunitinib daily at 40 mg/kg orally. The combination of sunitinib with OSI-027 at 12.5, 25 and 50 mg/kg resulted in significant TGI of 100% with 22%, 47% and 58% tumor regressions respectively. The combinations were well tolerated with minimal body weight loss (1-3%). Therefore, OSI-027 in combination with sunitinib demonstrated therapeutic synergy with improved tumor growth inhibition and tumor regressions compared with treatment by single agent alone.

TABLE 3

| | Avg. | % | | Morbidity |
|---|---|---|---|---|
| Drug | TGI | Reg. | BWL | (Mortality) |
| OSI-027, 50 mg/kg qd | 83% | 8% | 0% | 0/8 |
| Sutent, 40 mg/kg qd | 55% | 0% | 0% | 0/8 |
| Sutent + OSI-027, 12.5 mg/kg qd | 100% | 22% | 1% | 0/8 |
| Sutent + OSI-027, 25 mg/kg qd | 100% | 47% | 1% | 0/8 |
| Sutent + OSI-027, 50 mg/kg qd | 100% | 58% | 3% | 0/8 |

Study No. 4: In vivo efficacy study of OSI-027 or sunitinib (Sutent ®) alone and the combination of OSI-027 with sunitinib (Sutent ®) in the OVCAR-5 ovarian carcinoma xenografts

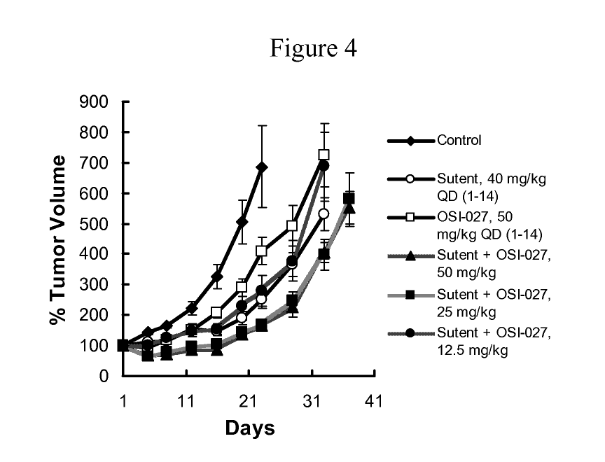

As shown in Table 4 and FIG. 4 below, daily oral administration of OSI-027 at 50 mg/kg for 14 days resulted in 78% mean tumor growth inhibition (TGI) as measured during the dosing period with 6% regression. Sunitinib (Sutent) as a single agent administered daily at 40 mg/kg for 14 days resulted in 71% TGI. In the combination groups, all mice received daily oral administration OSI-027 at 12.5, 25 or 50 mg/kg every day with sunitinib daily at 40 mg/kg orally. Combination of sunitinib with of OSI-027 at 25 and 50 mg/kg resulted in significant TGI of 100% with 37% and 33% regressions respectively. The combinations were well tolerated with minimal body weight loss (1-7%). Therefore, OSI-027 in combination with sunitinib demonstrated therapeutic synergy with improved tumor growth inhibition and tumor regressions compared with treatment by single agent alone.

TABLE 4

| | Avg. | % | | Morbidity |
|---|---|---|---|---|
| Drug | TGI | Reg. | BWL | (Mortality) |
| OSI-027, 50 mg/kg qd | 78% | 6% | 0% | 0/8 |
| Sutent, 40 mg/kg qd | 71% | 0% | 0% | 0/8 |
| Sutent + OSI-027, 12.5 mg/kg qd | 65% | 0% | 0% | 0/8 |
| Sutent + OSI-027, 25 mg/kg qd | 100%* | 37% | 1% | 0/8 |
| Sutent + OSI-027, 50 mg/kg qd | 100%* | 33% | 7% | 0/8 |

Study No. 5: In vivo efficacy study of OSI-027 or bevacizumab (Avastin ®) alone and in combination of OSI-027 with bevacizumab (Avastin ®) in SKOV3 ovarian carcinoma xenografts.

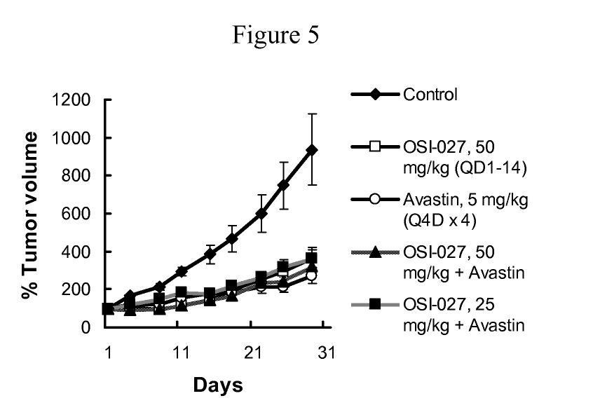

As shown in Table 5 and FIG. 5 below, daily oral administration of OSI-027 at 50 mg/kg for 14 days resulted in 86% mean tumor growth inhibition (TGI) as measured during the dosing period with 4% tumor regression. Bevacizumab as a single agent administered at 5 mg/kg q4d×4 resulted in 67% TGI with 0% tumor regression. In the combination groups, all mice received daily oral administration OSI-027 at 25 mg/kg or 50 mg/kg every day with bevacizumab at 5 mg/kg q4d×4 intravenously. Combination of bevacizumab with OSI-027 at 25 mg/kg resulted in a mean TGI of 60%. Combination of bevacizumab with OSI-027 at 50 mg/kg resulted in 83% TGI and 12% regression. The combinations were well tolerated with minimal body weight loss (0-4%).

TABLE 5

| | Avg. | % | | Morbidity |
|---|---|---|---|---|
| Drug | TGI | Reg. | BWL | (Mortality) |
| OSI-027, 50 mg/kg qd | 86% | 4% | 2% | 0/8 |
| Avastin, 4 mg/kg q4d | 67% | 0% | 0% | 0/8 |
| Avastin + OSI-027, 25 mg/kg qd | 60% | 0% | 0% | 0/8 |
| Avastin + OSI-027, 50 mg/kg qd | 83% | 12% | 4% | 0/8 |

### DEFINITIONS

The term “cancer” in an animal refers to the presence of cells possessing characteristics typical of cancer-causing cells, such as uncontrolled proliferation, immortality, metastatic potential, rapid growth and proliferation rate, and certain characteristic morphological features. Often, cancer cells will be in the form of a tumor, but such cells may exist alone within an animal, or may circulate in the blood stream as independent cells, such as leukemic cells.

“Cell growth”, as used herein, for example in the context of “tumor cell growth”, unless otherwise indicated, is used as commonly used in oncology, where the term is principally associated with growth in cell numbers, which occurs by means of cell reproduction (i.e. proliferation) when the rate the latter is greater than the rate of cell death (e.g. by apoptosis or necrosis), to produce an increase in the size of a population of cells, although a small component of that growth may in certain circumstances be due also to an increase in cell size or cytoplasmic volume of individual cells. An agent that inhibits cell growth can thus do so by either inhibiting proliferation or stimulating cell death, or both, such that the equilibrium between these two opposing processes is altered.

“Tumor growth” or “tumor metastases growth”, as used herein, unless otherwise indicated, is used as commonly used in oncology, where the term is principally associated with an increased mass or volume of the tumor or tumor metastases, primarily as a result of tumor cell growth.

“Abnormal cell growth”, as used herein, unless otherwise indicated, refers to cell growth that is independent of normal regulatory mechanisms (e.g., loss of contact inhibition). This includes the abnormal growth of: (1) tumor cells (tumors) that proliferate by expressing a mutated tyrosine kinase or over-expression of a receptor tyrosine kinase; (2) benign and malignant cells of other proliferative diseases in which aberrant tyrosine kinase activation occurs; (4) any tumors that proliferate by receptor tyrosine kinases; (5) any tumors that proliferate by aberrant serine/threonine kinase activation; and (6) benign and malignant cells of other proliferative diseases in which aberrant serine/threonine kinase activation occurs.

The term “treating” as used herein, unless otherwise indicated, means reversing, alleviating, inhibiting the progress of, or preventing, either partially or completely, the growth of tumors, tumor metastases, or other cancer-causing or neoplastic cells in a patient. The term “treatment” as used herein, unless otherwise indicated, refers to the act of treating.

The phrase “a method of treating” or its equivalent, when applied to, for example, cancer refers to a procedure or course of action that is designed to reduce or eliminate the number of cancer cells in an animal, or to alleviate the symptoms of a cancer. “A method of treating” cancer or another proliferative disorder does not necessarily mean that the cancer cells or other disorder will, in fact, be eliminated, that the number of cells or disorder will, in fact, be reduced, or that the symptoms of a cancer or other disorder will, in fact, be alleviated. Often, a method of treating cancer will be performed even with a low likelihood of success, but which, given the medical history and estimated survival expectancy of an animal, is nevertheless deemed an overall beneficial course of action.

The term “therapeutically effective agent” means a composition that will elicit the biological or medical response of a tissue, system, animal or human that is being sought by the researcher, veterinarian, medical doctor or other clinician.

The term “therapeutically effective amount” or “effective amount” means the amount of the subject compound or combination that will elicit the biological or medical response of a tissue, system, animal or human that is being sought by the researcher, veterinarian, medical doctor or other clinician.

The term “administered” or “administering” as used herein is meant parenteral and/or oral administration. By “parenteral” is meant intravenous, subcutaneous and intramuscular administration.

The term “synergistic effect” or “synergy” as used herein means that the therapeutic effect (e.g., inhibition of tumor cell growth, and/or tumor regression) of a combination comprising two or more agents is more effective than the therapeutic effect of a treatment where only a single agent alone is applied. For instance, the inhibition of tumor cell growth by the combination treatment is more effective than that of the each agent of the combination when applied alone. Further, a synergistic effect of a combination of two or more agents permits the use of lower dosages of one or more of the agents and/or less frequent administration of said agents to a patient having tumors or tumor metastases. The ability to utilize lower dosages of an agent and/or to administer said agent less frequently reduces the toxicity associated with the administration of said agent to a patient without reducing the efficacy of said agent in the prevention, management or treatment of tumors or tumor metastases. In addition, a synergistic effect can result in improved efficacy of agents in the prevention, management or treatment of tumors or tumor metastases. Moreover, a synergistic effect of a combination of two or more agents may avoid or reduce adverse or unwanted side effects associated with the use of either agent alone.

The term “refractory” as used herein is used to define a cancer for which treatment (e.g. chemotherapy drugs, biological agents, and/or radiation therapy) has proven to be ineffective. A refractory cancer tumor may shrink, but not to the point where the treatment is determined to be effective. Typically however, the tumor stays the same size as it was before treatment (stable disease), or it grows (progressive disease).

The term “progression-free survival time” (“PFST”) as used herein means the time from initiation of the treatment in accordance with the present invention to the documentation of disease progression or recurrence by histological or cytological evidence.

For purposes of the present invention, “co-administration of and “co-administering” an mTOR inhibitor and an angiogenesis inhibitor refer to any administration of the two active agents, either separately or together, where the two active agents are administered as part of an appropriate dose regimen designed to obtain the benefit of the combination therapy. Thus, the two active agents can be administered either as part of the same pharmaceutical composition or in separate pharmaceutical compositions. The mTOR inhibitor can be administered prior to, at the same time as, or subsequent to administration of the angiogenesis inhibitor, or in some combination thereof. Likewise, the angiogenesis inhibitor can be administered prior to, at the same time as, or subsequent to administration of the mTOR inhibitor, or in some combination thereof.

1-26. (canceled)

27. A method of treating a tumor or tumor metastasis comprising treating a patient in need thereof with a regimen comprising administering OSI-027 and an angiogenesis inhibitor, wherein the regimen provides a therapeutically effective synergistic or additive effect.

28. The method of claim 27, wherein the mTOR inhibitor comprises a tromethamine salt of OSI-027.

29. The method of claim 28, wherein the mTOR inhibitor and the angiogenesis inhibitor behave synergistically.

30. The method of claim 29, wherein the angiogenesis inhibitor comprises an inhibitor of VEGF activity.

31. The method of claim 29, wherein the angiogenesis inhibitor comprises bevacizumab, aflibercept, ABT-869, axitinib, ranibizumab, or BIBF1120.

32. The method of claim 29, wherein the angiogenesis inhibitor comprises an anti-VEGF antibody.

33. The method of claim 29, wherein the angiogenesis inhibitor comprises bevacizumab or ranizumab.

34. The method of claim 29, wherein the angiogenesis inhibitor comprises a VEGFR inhibitor.

35. The method of claim 29, wherein the angiogenesis inhibitor comprises sunitinib, sorafenib, vatalanib, AMG-706, CP-547632, pazopanib, ABT-869, IMC-1C11, cediranib, KM-2550, 2C3, IMC-18F1, or OSI-930.

36. The method of claim 29, wherein the angiogenesis inhibitor comprises sunitinib.

37. The method of claim 29, wherein the tumor or tumor metastasis is insensitive or refractory to treatment with the angiogenesis inhibitor as a single agent.

38. The method of claim 29, which results in improved survival of the patient as compared to treatment with either the mTOR or the angiogenesis inhibitor alone.

39. The method of any one of claim 29, wherein the mTOR pathway is activated in the tumor.

40. The method of claim 29, wherein the tumor comprises ovarian, prostate, or non small cell lung cancer.

41. The method of claim 29, wherein the tumor comprises renal cell carcinoma, endometrial carcinoma, or glioblastoma.

42. The method of claim 29, wherein the tumor comprises lymphoma or pancreatic cancer.

43. The method of claim 29, wherein OSI-027 is administered in an amount of about 0.5 to about 5 mg/kg body weight on days of administration.

44. The method of claim 29, wherein OSI-027 and bevacizumab are administered in a mass ratio of about 6 to about 15.

45. The method of claim 29, wherein OSI-027 and sunitinib are administered in a mass ratio of about 0.2 to about 2.
